(19) United States
(12) Patent Application Publication
(10) Pub. No.: US 2006/0085044 A1
(43) Pub. Date: Apr. 20, 2006

(54) Electromedical implant

(75) Inventors: Jurgen Drews, Pirna (DE); Steffen Hickmann, Heidenau (DE); Dipl-Chem Gerd Fehrmann, Pirna (DE); Wiebke Neumann, Berlin (DE); Roland Staub, Borggiesshubel (DE); Werner Uhrlandt, Berlin (DE)

Correspondence Address: HAHN LOESER & PARKS, LLP, One GOJO Plaza, Suite 300, AKRON, OH, 44311-1076, US

(73) Assignee: Biotronik CRM Patent AG

(21) Appl. No.: 11/244,440

(22) Filed: Oct. 5, 2005

(30) Foreign Application Priority Data
Oct. 12, 2004 (DE) 102004049 778.8
Dec. 6, 2004 (DE) 102004059096.6
Apr. 20, 2005 (DE) 102005018128.7

(51) Int. Cl. A61N 1/375

(52) U.S. Cl. 607/36

(57) ABSTRACT

An electromedical implant is disclosed comprising a housing which is hermetically sealed off to the outside; a power supply unit comprising a first shell with a first electrically conductive main surface and a first side wall, and a second shell comprising a second main surface and a second side wall which is embedded into the housing that is hermetically sealed off to the outside; a control unit that is electrically connected to the power supply unit; a header for contacting electrode lines, feedthroughs for leading away therapeutic pulses or pulse sequences from the housing that is hermetically sealed off to the outside. In this embodiment the electrical control unit is electrically connected to the power supply unit in a two-pole arrangement; the second main surface of the power supply unit has at least 0.7 times the surface of the base surface of the housing of the electromedical implant, which housing is hermetically sealed off to the outside; and the height of the power supply unit is at most 0.5 times the height of the housing of the electromedical implant, which housing is hermetically sealed off to the outside.

### CROSS-REFERENCE TO RELATED APPLICATIONS/INCORPORATION BY REFERENCE

This U.S. patent application claims priority to and claims the benefit of the following German patent applications:
DE 10 2004 049 778.8, filed Oct. 12, 2004;
DE 10 2004 059 096.6, filed Dec. 6, 2004; and
DE 10 2005 018 128.7, filed Apr. 20, 2005.

### TECHNICAL FIELD

Certain embodiments of the present invention relate to electromedical implants. More particularly, certain embodiments of the present invention relate to an electromedical implant with a power supply unit for easy and economical production.

### BACKGROUND OF THE INVENTION

Intercardiac therapy has developed into a standard procedure that has proven itself millions of times. In this process an electromedical implant is implanted in a skin pocket of a patient undergoing therapy, and is for example permanently electrically connected to the heart by way of an electrode line. Such electromedical implants include cardiac pacemakers, implantable defibrillators, medication pumps, neurostimulators or any other device that emits electrical power and is implanted in a human or animal body.

Optimal space-saving utilization of space in the limited space available within the housing of such an implant is the big challenge that presents itself in an electromedical implant. Up to now, electromedical implants are made in a side-by-side design, where the individual components of such an electromedical implant are arranged side-by-side on the base surface. For example, the power supply unit is located on the base surface of an electromedical implant, beside the electrical control unit of said electromedical implant. This design is associated with a serious disadvantage in that the manufacture of an electromedical implant in side-by-side construction so as to meet the above-mentioned requirements concerning utilization of space requires very considerable manual effort.

For this reason efforts have been made early to simplify and automate the production of such devices, accompanied by simplification and standardization of the design of pacemakers. This simplification naturally also relates to production so that an electromedical implant can be produced at significantly lower cost. In this approach the bottom-up design, i.e. a design where the components of the electromedical implant are installed one on top of the other, has been shown to be very advantageous. In this arrangement the large components are installed first with the lighter and smaller components then being placed onto said large components. The power supply unit continues to be the largest component because safe, secure and enduring supply of electrical power is a very important aspect of an electromedical implant. These requirements are due to the need to provide the patient with the best-possible convenience, including a minimum of after-care examinations. These power supply units are therefore designed to provide the longest possible service life. Unfortunately the capacity of power supply units is related to their volume, and for this reason the power supply unit will for an unforeseeable time remain the largest component of an electromedical implant, and therefore will remain the lowermost component in a bottom-up design. In the context of this document, the term power supply unit refers to all batteries, storage batteries or other known power generating devices.

A hermetically-sealed battery 1 as shown in FIG. 1 is one example of a power supply unit of the above-mentioned side-by-side design. The semi-oval battery comprises a housing 2 and a cover 3. Furthermore, the feedthrough 4 is shown on the cover 3, which comprises a feedthrough pin 5 and a bush 6 visible from the outside. As a rule, glass-metal feedthroughs are used for this, whose metal bush is welded into a borehole of the cover. If the battery is filled with a liquid, gel-like or polymer electrolyte, usually the cover 3 of the battery 1 comprises an aperture which is hermetically sealed after the filling process. For this purpose, as a rule a sealing piece is welded in or riveted in.

FIG. 2 also shows a design, known from the state of the art, of an electromedical implant 7. The power supply unit 1 is embedded in a precise fit in the hermetically sealed housing 8 of the electromedical implant 7. In the hermetically sealed housing 8 of the electromedical implant 7 the electronic control unit 9 is arranged above the power supply unit 1 and is electrically connected to the power supply unit 1 by way of the feedthrough pin 5. Such a power supply unit is associated with a disadvantage in that the position of the feedthrough on the flat side of a power supply unit does not allow a cost effective bottom-up design. An example of such a power supply unit is shown in U.S. Pat. No. 4,830,940.

Patent specification U.S. Pat. No. 6,613,474 B2 describes a flat battery which is based on joining two metal housing half-shells of precise fit. Both housing parts are joined with precise fit so as to facilitate hermetically sealed welding. This invention, too, is associated with a disadvantage in that the position of the feedthrough at a flat side prevents a cost-effective bottom-up design.

WO 02/32503 A1 describes an electromedical implant with a battery. According to said publication the implantable device comprising a battery part and an electronics part is designed such that at least one face of the power supply unit forms the outside of the electromedical implant. This represents a quasi bottom-up design because the large component is simply attached to the smaller components. However, this design is associated with a very substantial disadvantage in that part of the housing of the power supply unit at the same time serves as the external housing of an electromedical implant. Should there be any leakage of the battery unit in the housing, the patient could suffer very series toxic effects.

One example of a bottom-up design is shown in EP 1 407 801 A2. The control unit of an electromedical implant is built onto a power supply unit which comprises a flat side, a bottom and a circumferential narrow side. This makes it possible to produce the implantable device in a single bottom-up design because the control unit of the implantable device can be installed on the flat side of the power supply unit.

This method is advantageous in that it provides optimum use of the available volume, which is limited by the housing of the electromedical implant.

Further limitations and disadvantages of conventional, traditional, and proposed approaches will become apparent to one of skill in the art, through comparison of such systems and methods with the present invention as set forth in the remainder of the present application with reference to the drawings.

### BRIEF SUMMARY OF THE INVENTION

Certain embodiments of the present invention avoid the above-mentioned disadvantages and provide an electromedical implant with a power supply unit that may be produced in the economical bottom-up design. The electromedical implant comprises a housing that is hermetically sealed off to the outside, and an advantageous power supply unit comprising a first shell with a first electrically conductive main surface and a first side wall, and a second shell comprising a second main surface and a second side wall. The power supply unit is embedded in the housing that is hermetically sealed off to the outside. An electrical control unit is electrically connected to the power supply unit. It has been shown to be particularly advantageous to electrically connect the electrical control unit in a two-pole arrangement by way of the first main surface of the power supply unit and to adapt the dimensions of the base surface of the power supply unit both in form and in shape to the base surface of the electromedical implant. This makes possible simple positioning of the power supply unit in the housing of the electromedical implant and prevents faulty or incorrect installation of the power supply unit in the electromedical implant.

The flat first main surface makes possible direct attachment of the control unit. The first main surface is designed such that it provides sufficient space to install the control unit. The first main surface comprises a glass-metal feedthrough, a filler aperture and contact elements which make bottom-up installation possible and which are arranged such that direct electrical contact of the electrical control unit is possible. The glass-metal feedthrough and the filler aperture are installed flush in the first main surface, which makes possible absolutely flat installation of the control unit. The power supply unit uses a special thrust piece that contributes to the stability of the internal design of the power supply unit. In the power supply unit a special retaining ring is used which considerably simplifies the use of complex geometries and at the same time contributes to the stability of the internal design of the power supply unit. In the power supply unit a special conductive metal discharge strip is used which establishes an electrically conductive connection between the pin of the glass-metal feedthrough and the conductive discharge grid of the electrode. This conductive metal discharge strip simplifies production of the power supply unit and simplifies contacting of electrodes that involve a complex geometry. Swelling of the power supply unit can be prevented by using the first main surface with an angled-off geometry; the mechanical stability can be improved in this way too.

These and other advantages and novel features of the present invention, as well as details of illustrated embodiments thereof, will be more fully understood from the following description and drawings.

### BRIEF DESCRIPTION OF SEVERAL VIEWS OF THE DRAWINGS

FIG. 1 illustrates a power supply unit from the state of the art.

FIG. 2 illustrates a power supply unit installed in an electromedical implant from the state of the art.

FIG. 3 illustrates a sectional aspect of a power supply unit installed in an electromedical implant, in accordance with an embodiment of the present invention.

FIG. 4 illustrates an exploded view of a power supply unit, in accordance with an embodiment of the present invention.

FIGS. 5A to 5D illustrate sectional aspects of embodiments showing the geometry of a power supply unit, in accordance with an embodiment of the present invention.

FIG. 6 illustrates design of the side walls during hermetic welding, in accordance with an embodiment of the present invention.

FIGS. 7A and 7B illustrate embodiments of the present invention relating to the stability and the prevention of swelling of a power supply unit in lateral view.

FIG. 8 illustrates contact elements installed on the first main surface of a power supply unit, in accordance with an embodiment of the present invention.

FIG. 9 illustrates a first main surface, divided into two, for contacting two poles, in accordance with an embodiment of the present invention.

FIG. 10 illustrates a sectional view of a glass-metal feedthrough, in accordance with an embodiment of the present invention.

FIG. 11 illustrates the interior structure of a power supply unit comprising a tension frame, in accordance with an embodiment of the present invention.

FIG. 12 illustrates a sectional view of the interior structure of a power supply unit comprising a glass-metal feedthrough and a filler aperture, in accordance with an embodiment of the present invention.

FIG. 13 illustrates a metal contact strip, in accordance with an embodiment of the present invention.

FIGS. 14A and 14B illustrate options for installing a metal contact strip, in accordance with an embodiment of the present invention.

FIGS. 15A to 15C illustrate installing the metal contact strip, in accordance with an embodiment of the present invention.

FIG. 16 illustrates a completely installed power supply unit, in accordance with an embodiment of the present invention.

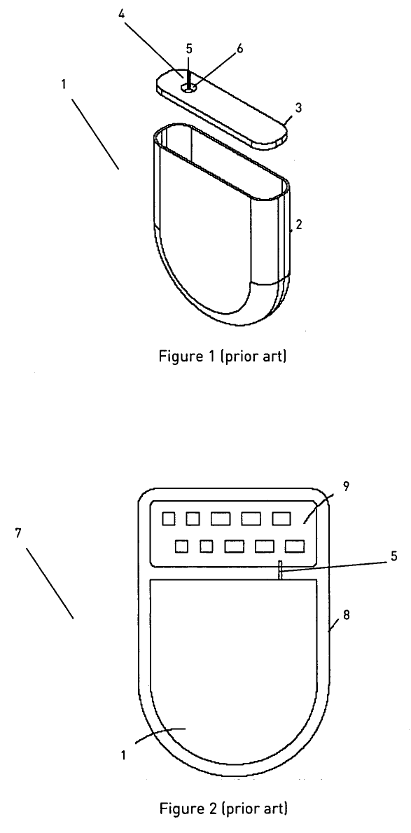

### DETAILED DESCRIPTION OF THE INVENTION

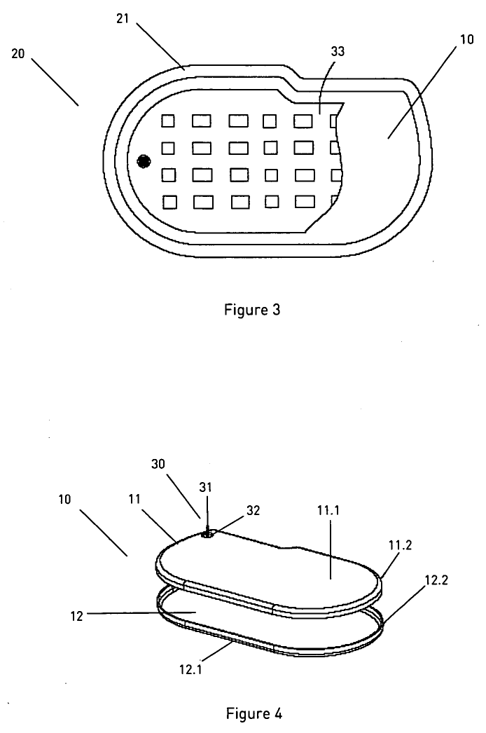

FIG. 3 shows the installed state of an electromedical implant 20. The housing 21, which is hermetically sealed off to the outside, the power supply unit 10 and the electrical control unit 33, which is installed on the first main surface 11.1 of the power supply unit, are shown. The first main surface 11.1 is designed such that it can accommodate the electrical control unit 33 of the implantable device 20. The size relationship between the base surface of the housing 21 that is hermetically sealed off to the outside and of the second main surface 12.1 of the power supply unit 10 is particularly favorable. In this arrangement the second main surface 12.1 of the power supply unit 10 has at least 0.7 times the surface of the base surface of the housing 21 of the electromedical implant 20. The following is favorable: 0.7 to 0.99 times the area, preferably 0.7 times to 0.95 times, particularly favorably 0.8 times to 0.9 times and particularly preferably 0.8 times to 0.85 times the size of the surface of the second main surface 12.1 in relation to the base surface of the housing 21. The height must not exceed 0.7 times the height of the housing 21 that is hermetically sealed off to the outside. The height of the power supply device 10 is favorably between 0.4 to 0.6 times, preferably between 0.5 and 0.6 times and particularly preferably between 0.55 and 0.6 times the height of the housing 21. This makes it possible to simply position the power supply unit 10 in the housing 21 of the electromedical implant 20 and prevents any erroneous faulty or incorrect installation of the power supply device 10 in the electromedical implant 20. In this arrangement space utilization of the power supply unit 10 is as efficient as possible so as to ensure good energy density of the power supply unit and thus a long service life of the finished device.

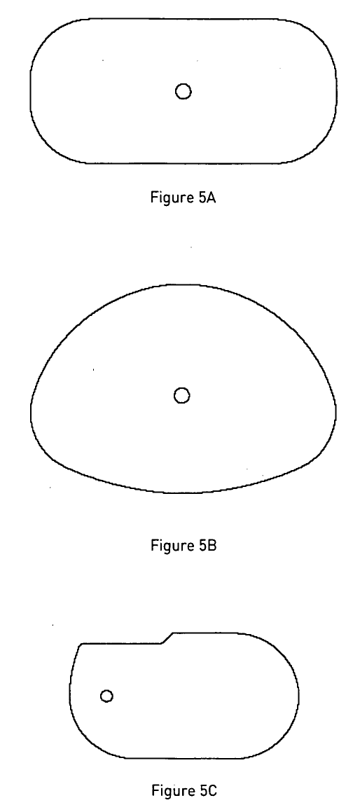

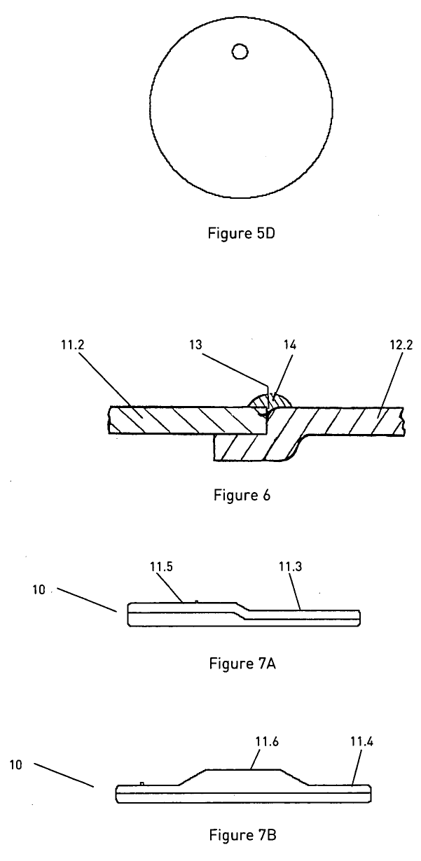

FIG. 4 shows a power supply unit device 10 of a flat design. The power supply unit 10 comprises a first shell 11 with a first main surface 11.1 and a sidewall 11.2, and a second shell 12 with a second main surface 12.1 and a second side wall 12.2. Both shells 11 and 12 with their side walls 11.2 and 12.2 are formed so that when their side walls are joined a form closure results. In this process a groove is formed which makes possible hermetic sealing of the two shells. The first main surface 11.1 has a glass-metal feedthrough 30 which comprises a feedthrough pin 31, a bush 32 and glass insulation. The second main surface 12.1 of the power supply unit 10 is situated on the interior surface of the hermetically sealed housing 21 of the electromedical implant 20. The form of the second main surface 12.1 corresponds to the base surface of the hermetically sealed housing 21 of the electromedical implant 20. Round, oval or physiologically shaped housing shapes can be implemented. Aspects of possible shapes of the main surfaces are shown in FIGS. 5A to 5D. Additional other shapes are also possible.

To also simplify production of the power supply unit 10, the two side walls 11.2 and 12.2 of the shells 11 and 12 are designed such that joining is very easy. FIG. 6 shows an exemplary design of the side walls 11.2 and 12.2 just before the welding process. The side walls 11.2 and 12.2 establish a form closure. At the contact face between the side walls 11.2 and 12.2 a circumferential groove 13 forms, to which a hermetically sealing weld seam 14 can be applied, for example using a laser welding process.

During operation of the power supply unit it can happen in various states that swelling of the power supply unit occurs. Excessive swelling can push the control unit that is affixed to the power supply unit against the inside of the hermetically sealed housing of the electromedical implant. This can lead to damage to the control unit. For this reason, any such swelling must be kept to a minimum.

It has been shown that a power supply unit with at least two different height levels is advantageous to prevent such swelling. As shown by way of example in FIGS. 7A and 7B the first main surface 11.1 of a power supply unit 10 comprises a first level 11.3 or 11.4 and a second level 11.5 or 11.6. Between the first level 11.3 or 11.4 and the corresponding second level 11.5 or 11.6 there is an angle which is between 30° and 60°, preferably between 40° and 50°, and particularly advantageously 45°. In order to achieve homogeneous use of space in the interior of the hermetically sealed housing 21 of the electromedical implant 20, the difference in height between the first level 11.3 or 11.4 and the second level 11.5 or 11.6 must equal the height of the control unit 33 attached to the first level 11.3 and 11.4.

For an implantable device a permanent electrically conductive connection between the power supply unit and the electronic control unit is of special importance. In a great many lithium batteries the housing is connected to the anode so as to be electrically conductive and can be contacted from the outside directly at the housing. Preferably, a reliable contact is made by welding or soldering. There is a disadvantage in that during direct welding or soldering of the control unit to the housing of the power supply unit damage can occur, e.g. as a result of inadvertent opening of the housing (loss of hermetic sealing) or as a result of damage to component assemblies resulting from heat input into the power supply unit.

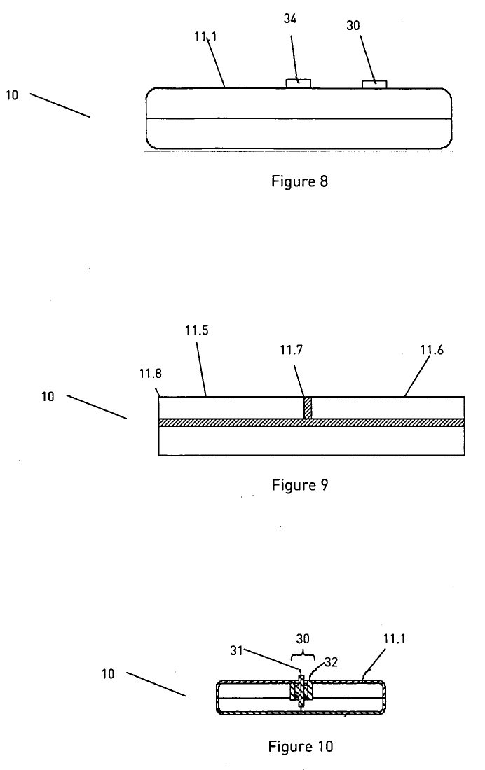

This disadvantage is for example eliminated as shown in FIG. 8. One or several contact elements 34 are integrated into the first main surface 11.1 of the power supply unit 10. The contact element 34 is connected to the first main surface 11.1 before the power supply unit 10 is hermetically sealed. Thus any damage to the integrity of the housing of the power supply unit 10 can be effectively precluded by way of a leakage test prior to closing the power supply unit 10. The contact element 34 is designed as flat as the glass-metal feedthrough 30 so that said contact element 34 does not lead to an increase in the height of the power supply unit 10 in conjunction with the control unit 33. The material thickness of the contact piece 34 is selected such that welding or soldering on the contact piece 34 is possible without in the process damaging the housing of the power supply unit 10 or the glass-metal feedthrough 30. Establishing a conductive mechanically stable connection (e.g. by means of welding or soldering) between the control unit 33 of the medical electrical device 20 and the first main surface 11.1 of the power supply unit 10 is considerably facilitated by the contact element 34, and the danger of damaging the power supply unit 10 as a result of heat input during establishment of the electrical contact is eliminated.

While the anode is tapped by way of the first main surface 11.1, the cathode of the power supply unit 10 is tapped by way of a pin 31 (in FIG. 3) of the glass-metal feedthrough 30. Furthermore, it is however also possible to lead both the anode and the cathode from the power supply unit 10 by way of glass-metal feedthroughs. This provides the advantage that the first shell 11 does not electrically contact the first main surface 11.1 of the power supply unit 10 and that consequently no insulation between the power supply unit 10 and the housing 21 of the electromedical implant 20 needs to be provided.

A further embodiment is shown in FIG. 9, which shows a power supply unit 10 with a first shell 11 and a second shell 12. The first shell 11 has two mutually insulated surfaces 11.5 and 11.6, which are mutually insulated with an insulation layer 11.7. Likewise, an insulation layer 11.8 can be provided between the first shell 11 and the second shell 12. It is thus possible to contact in a two-pole arrangement both the cathode and the anode by way of the main surface 11.1, which in this case comprises the surfaces 11.5, 11.6 and the insulation layer 11.7. This results in advantages in simplifying the electrical connection between the power supply unit 10 and the electrical control unit 33 of the electromedical implant 20. In this design variant the contact element 34 shown in FIG. 8 is also used, however, obviously at least one contact element for each partial surface 11.5 and 11.6 is used.

FIG. 10 shows the integration of a glass-metal feedthrough 30 in the first main surface 11.1 of the power supply unit 10. At the pin 31 of the glassmetal feedthrough a pole of the power supply unit 10 is led from the inside of the power supply unit 10 to the outside so as to be insulated from the metal of the first main surface 11.1. The glass-metal feedthrough 30 ensures hermetic sealing of the power supply unit 10. Particularly advantageous is the special installation of the glass-metal feedthrough 30 wherein the bush 32 of the glass-metal feedthrough 30 is welded into the first main surface 11.1 so as to be flush on the outside. This ensures that the bush 32 of the feedthrough 30 does not protrude beyond the outer surface of the first main surface 11.1. The pin 31 of the glass-metal feedthrough 30 is designed to be so short that it just protrudes sufficiently beyond the first main surface 11.1 of the power supply unit 10 to ensure electrically conductive contacting of the control unit 33. This design has an advantage in that in this way a particularly flat space-saving arrangement of the power supply unit 10 and of the control unit 33 in the housing 21 of the implantable device 20 becomes possible.

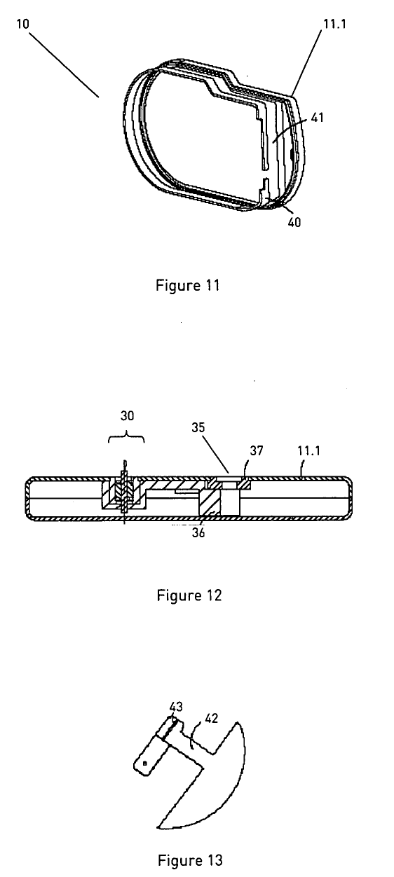

FIG. 11 shows a solution which makes it possible also with complex geometries to maintain the distance between the electrodes and the separator, and thus the distance between the electrodes themselves, as precisely as possible. This specification is met by the use of a special retaining ring 40 in a power supply unit, which retaining ring tensions the separator firmly and homogeneously onto the electrode of the power supply unit. This retaining ring 40 is preferably made from a special plastic material which is inert vis-a-vis other components of the power supply unit (e.g. vis-a-vis the electrolyte) while at the same time being an electrical insulator. Preferably suitable are polyethylene and polypropylene as well as polyhalogenated olefins, which can be thermoplastically formed (e.g. TEFLON®, HALAR®, KYNAR® or SOLEF®). If the separator acts so as to be electrically insulating, the retaining ring 40 can also be placed below the separator. In this case the retaining ring 40 can also be made from a metal which is inert vis-a-vis other components of the power supply unit, e.g. from stainless steel. The abovementioned retaining ring 40 comprises an aperture 41 through which the conductive discharge line of the cathode can be fed. This special design characteristic makes possible a particularly simple and space-saving connection of the cathode to the glass-metal feedthrough 30 situated on the first main surface 11.1 of the power supply unit 10, since the conductive discharge line of the cathode does not have to be bent or be routed to the glass-metal feedthrough 30 so as to be perpendicular to the main axis of the power supply unit.

FIG. 12 shows that a filler aperture 35 is integrated in the first main surface 11.1 of the power supply unit 10. The power supply unit 10 is filled with liquid or gel-like electrolyte through the filler aperture 35. The particular design of the filler aperture 35 provides a special advantage. Below the filler aperture 35 there is a special thrust piece 36 which together with the retaining ring 40 ensures the stability of the interior construction of the cell. Furthermore, this thrust piece 36 is designed such that it makes possible the welding-in of a flush-fitting cap 37 which hermetically seals the power supply unit after the filling procedure. The flush-fitting cap 37, which does not protrude from the first main surface 11.1 of the power supply unit 10, ensures flat space-saving installation of the control unit 33 on the power supply unit 10.

In the filling procedure the power supply unit is evacuated. Electrolyte is sucked in by the negative pressure in the cell (vacuum filling). In the case of flat power supply units this procedure is particularly difficult because the main sides of the power supply unit can become deformed as a result of external pressure. Placement of the filler nozzle is another technical problem that has to be solved. Said filler nozzle must be pressed over the filler aperture with a seal so as to effectively prevent air ingress during evacuation. In this process there is a risk of the housing of the power supply unit becoming deformed as a result of the filler nozzle being pressed on. The region of the filler aperture of the power supply unit is also mechanically loaded when the sealing cap is pushed in.

The thrust piece 36 stabilizes the geometry of the power supply unit 10, in particular in the region of the filler aperture 35. Consequently the filler nozzle can be pressed on at greater pressure (better sealing action). At the same time deformation of the housing of the power supply unit 10 during evacuation and deformation during pressing-on of the sealing cap 37 is prevented. Due to the special shape of the thrust piece 36 the mechanically sensitive glass-metal feedthrough 30 is enclosed at the same time and thus additionally protected against mechanical damage. This also makes it possible to place the filler aperture 35 in direct proximity to the glass-metal feedthrough 30.

The retaining ring 40 also stabilizes the geometry of the power supply unit 10 because said retaining ring 40 fills the space between the inside of the first and second main surfaces 11.1 and 12.1 and the electrodes. In this way the inner componentry of the power supply unit 10 is mechanically fixed so that it cannot slide out of place. Complete filling of the space outside the electrodes additionally stabilizes the power supply unit 10 because no free space is available within, into which free space the housing could deform, for example during evacuation or filling.

On the inside a metal contact strip 42 is welded to pin 31 of the glass-metal feedthrough 30 (FIG. 13). This metal contact strip 42 comprises a gap 43. The conductive discharge grid fed out from the electrode is placed onto the gap 43 and through the gap 43 is welded to the metal backing. This design simplifies the welding process because the materials do not have to be butt welded to each other. The welding process is also simplified in that all the materials to be welded together are arranged in the main axis of the power supply unit. The construction of power supply units with complex electrode geometries is significantly simplified.

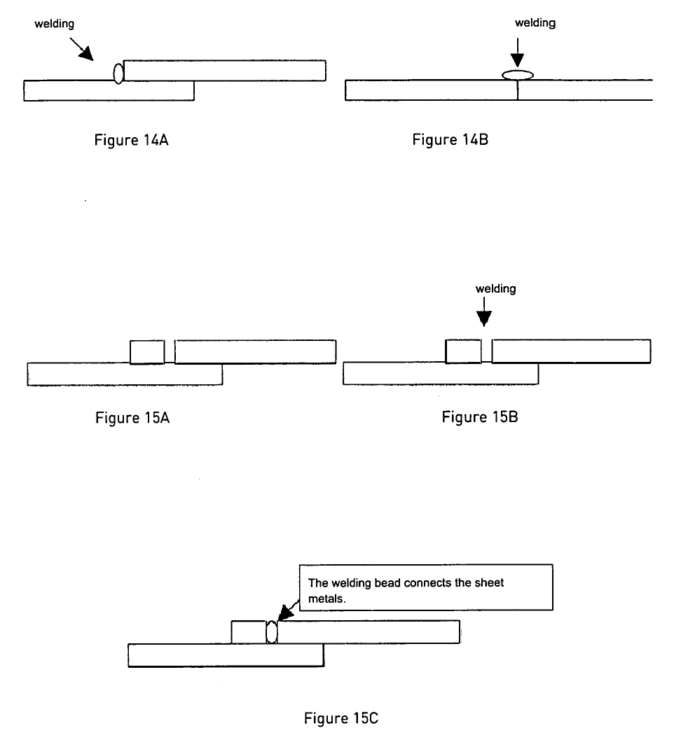

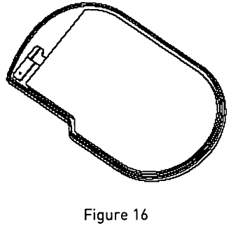

The conductive discharge grid of the electrode is placed onto the gap (FIG. 15A). The conductive discharge grid of the electrode and the conductive metal discharge strip are interconnected in one spatial plane by the gap (FIG. 15B/15C).

Butt welding (FIG. 14B) or edge welding (FIG. 14A) can be carried out as an alternative to this procedure.

The procedures of FIGS. 14A and 14B are significantly more complex because 14A requires precise inclined laser welding, while 14B requires very precise positioning of the components. The method is advantageous because of easy positioning of the components and because it offers the possibility of welding directly from the top.

The glass-metal feedthrough 30, the filler aperture 35 with sealing cap 37 and the contact elements 34 are positioned onto the first main surface 11.1 in such a way that an adequate surface for accommodating the control unit 33 is provided, while at the same time direct connection of the control unit 33 to the poles is ensured. As a result of positioning the contact elements 34 and the glass-metal feedthrough 30 on the first main surface 11.1, and as a result of their flat construction, the electromedical implant can be designed in one plane. This construction greatly simplifies contacting.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

What is claimed is:

1. An electromedical implant comprising:
a housing which is hermetically sealed off to the outside;
a power supply unit comprising a first shell with a first electrically conductive main surface and a first side wall, and a second shell comprising a second main surface and a second side wall which is embedded into the housing that is hermetically sealed off to the outside; and
a control unit that is electrically connected to the power supply unit, wherein the electrical control unit is electrically connected, by way of the first main surface, to the power supply unit in a two-pole arrangement, and
wherein the second main surface of the power supply unit has at least 0.7 times the surface of the base surface of the housing, and
wherein the height of the power supply unit is at most 0.5 times the height of the housing that is hermetically sealed off to the outside.

2. The electromedical implant of claim 1, wherein the first and the second shell of the power supply unit, with their two side walls, are formed such that when the shells are joined, a form closure results which makes possible easy hermetic welding.

3. The electromedical implant of claim 1, wherein a glass-metal feedthrough forms a first pole of the electrical connection on the first main surface of the power supply unit and wherein the first pole is substantially cathodic.

4. The electromedical implant of claim 2, wherein a glass-metal feedthrough forms a first pole of the electrical connection on the first main surface of the power supply unit and wherein the first pole is substantially cathodic.

5. The electromedical implant of claim 3, wherein the glass-metal feedthrough in the first main surface of the power supply unit comprises a bush.

6. The electromedical implant of claim 4, wherein the glass-metal feedthrough in the first main surface of the power supply unit comprises a bush.

7. The electromedical implant of claim 1, wherein a second pole of the electrical connection forms the first main surface of the power supply unit and wherein the second pole is substantially anodic.

8. The electromedical implant of claim 2, wherein a second pole of the electrical connection forms the first main surface of the power supply unit and wherein the second pole is substantially anodic.

9. The electromedical implant of claim 7 wherein the electrical connection of the second pole is made by way of contact elements on the electrically conductive first main surface of the power supply unit.

10. The electromedical implant of claim 8 wherein the electrical connection of the second pole is made by way of contact elements on the electrically conductive first main surface of the power supply unit.

11. The electromedical implant of claim 9 wherein the first main surface of the power supply unit comprises at least two different height levels wherein the difference in height between said two different height levels substantially equals a height of the control unit.

12. The electromedical implant of claim 10 wherein the first main surface of the power supply unit comprises at least two different height levels wherein the difference in height between said two different height levels substantially equals a height of the control unit.

13. The electromedical implant of claim 11, wherein a connection between the different height levels is at an angle which is between 30 degrees and 60 degrees.

14. The electromedical implant of claim 12, wherein a connection between the different height levels is at an angle which is between 30 degrees and 60 degrees.

15. The electromedical implant of claim 13, wherein the second main surface of the power supply unit is flat and has a shape substantially corresponding to the housing that is hermetically sealed off to the outside.

16. The electromedical implant of claim 14, wherein the second main surface of the power supply unit is flat and has a shape substantially corresponding to the housing that is hermetically sealed off to the outside.

17. The electromedical unit of claim 15, wherein the second main surface of the power supply unit is 0.7 to 0.99 times the area of the base surface of the housing of the electromedical implant.

18. The electromedical unit of claim 16, wherein the second main surface of the power supply unit is 0.7 to 0.99 times the area of the base surface of the housing of the electromedical implant.
